SEQUENCE LISTING (2 sequences)

SEQ ID NO: 1 - protein, 24 aa, Artificial
  A peptide having the structure of the p277 sequence of hsp60 in which one or both cysteine residues are replaced by valine has substantially the same biological activity
  MISC_FEATURE (6)..(6)
  MISC_FEATURE (11)..(11)
  MISC_FEATURE (19)..(19)
VLGGGXALLRXIPALDSLXPANED

SEQ ID NO: 2 - protein, 24 aa, Artificial
  A peptide having the structure of the p277 sequence of hsp60 in which one or both cysteine residues are replaced by valine has substantially the same biological activity
VLGGGVALLRVIPALDSLTPANED

(19) United States
(12) Patent Application Publication
(10) Pub. No.: US 2007/0299245 A1
(43) Pub. Date: Dec. 27, 2007

(54) P277 PEPTIDE FORMULATIONS OR VARIANTS THEREOF HAVING OPTIMIZED STABILITY

(75) Inventors: Caroline Hops, Hofheim (DE); Werner Mueller, Frankfurt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt (DE)

(21) Appl. No.: 11/678,792

(22) Filed: Feb. 26, 2007

Related U.S. Application Data
(63) Continuation of application No. PCT/EP05/09223, filed on Aug. 26, 2005.

(30) Foreign Application Priority Data
Sep. 10, 2004 (DE) 102004043750.5

(51) Int. Cl. C07K 14/00

(52) U.S. Cl. 530/325

(57) ABSTRACT

The invention relates to formulations containing the peptide p277 or variants thereof which are characterized by having increased stability during storage and transport.

### FIELD OF THE INVENTION

The invention relates to formulations containing the peptide p277 or variants thereof which are characterized by having increased stability during storage and transport.

### BACKGROUND OF THE INVENTION

The p277 peptide and its variants consist of 24 amino acids and have the structure Val-Leu-Gly-Gly-Gly-X₁-Ala-Leu-Leu-Arg-X₂-Ile-Pro-Ala-Leu-Asp-Ser-Leu-X₃-Pro-Ala-Asn-Glu-Asp (SEQ. I.D. No. 1), where X₁, X₂ may be Cys or Val and X₃ may be Thr or Lys. Said peptide is described in EP 0 820 303. Further formulations of p277 and its variants are disclosed in EP 0 837 672.

The p277 peptide and its variants are not stable at room temperature and must therefore be stored in the deep-freeze state below −10° C. The known formulations of p277 and its variants, which are the corresponding lyophilizates, are also not stable at room temperature and must therefore be stored and transported in the deep-freeze state. In order to achieve a stability of 12 months, said formulations must be permanently stored below −10° C.

It was the object of the invention to find formulations of the p277 peptide and its variants, which are stable at relatively high temperatures for at least 6 months. It was particularly desirable to find those formulations which are stable at refrigerator temperatures of from +2° C. to +8° C. or even at room temperature (from +15° C. to +25° C.). Stable means that the total proportion of secondary products does not increase by more than 1% over a period of 6 months.

It was intended, in particular, to find a stable formulation of this kind for p277 peptide (Val⁶-Val¹¹-Thr¹⁹) having the structure Val-Leu-Gly-Gly-Gly-Val-Ala-Leu-Leu-Arg-Val-Ile-Pro-Ala-Leu-Asp-Ser-Leu-Thr-Pro-Ala-Asn-Glu-Asp.

### SUMMARY OF THE INVENTION

The invention therefore relates to formulations which contain p277 or its variants having the structure Val-Leu-Gly-Gly-Gly-X₁-Ala-Leu-Leu-Arg-X₂-Ile-Pro-Ala-Leu-Asp-Ser-Leu-X₃-Pro-Ala-Asn-Glu-Asp (SEQ. I.D. No. 1), where X₁, X₂ may be Cys or Val and X₃ may be Thr or Lys, and a citrate buffer.

### DETAILED DESCRIPTION

Preference is given to formulations containing p277 (Val⁶-Val¹¹-Thr¹⁹) having the structure Val-Leu-Gly-Gly-Gly-Val-Ala-Leu-Leu-Arg-Val-Ile-Pro-Ala-Leu-Asp-Ser-Leu-Thr-Pro-Ala-Asn-Glu-Asp (SEQ. I.D. No. 2), and a citrate buffer.

The invention furthermore relates to formulations containing p277 or its variants, an excipient from the group consisting of trehalose, maltose, lactose and mannitol, and a citrate buffer.

Preference is given to formulations containing the excipient mannitol. Preference is furthermore given to a mannitol content of from 5 mg/ml to 200 mg/ml. Particular preference is given to a mannitol content of from 25 mg/ml to 50 mg/ml. Very particular preference is given to a mannitol content of 40 mg/ml.

Further preference is given to formulations containing p277 and its variants, mannitol and a citrate buffer, wherein the pH may be from pH 3 to pH 6.

Particular preference is given to formulations containing p277 and its variants, mannitol and a citrate buffer, wherein the pH may be from pH 4.5 to pH 6.

A citrate buffer means an aqueous solution of citrates, preferably alkali metal citrates, particularly preferably sodium citrate or potassium citrate, and a strong acid, preferably hydrochloric acid (HCl).

The formulations of the invention may be present in the form of a solution or a lyophilizate, preferably a lyophilizate.

The examples listed below are intended to illustrate but not limit the invention.

TABLE 1

| | Example | | | | | | | | | | | | Comparative | |
|---|---|---|---|---|---|---|---|---|---|---|---|---|---|---|
| | 1 | | 2 | | 3 | | 4 | | 5 | | 6 | | Example 1 | |
| p277 (Val⁶-Val¹¹-Thr¹⁹) | 1.3 | mg | 1.3 | mg | 1.3 | mg | 1.3 | mg | 1.3 | mg | 1.3 | mg | 1.3 | mg |
| Trehalose | 40.0 | mg | — | | — | | — | | — | | — | | | |
| Maltose | — | | 40.0 | mg | — | | — | | — | | — | | | |
| Lactose | — | | — | | 40.0 | mg | — | | — | | — | | | |
| Mannitol | — | | — | | — | | 40.0 | mg | 40.0 | mg | 40.0 | mg | 40.0 | mg |
| Citrate buffer pH 3.0* | — | | — | | — | | ad 1.0 | ml | — | | — | | | |
| Citrate buffer pH 4.5* | ad 1.0 | ml | ad 1.0 | ml | ad 1.0 | ml | — | | ad 1.0 | ml | — | | | |
| Citrate buffer pH 6.0* | — | | — | | — | | — | | — | | ad 1.0 | ml | | |
| Water for injection purposes | | | | | | | | | | | | | ad 1.0 | ml |

* Citrate buffer composition:

| NaOH | 8.000 | g |
|---|---|---|
| Citric acid * 1 H₂O | 21.056 | g |
| HCl | ad pH 3.0; pH 4.5; pH 6.0 | |
| Water for injection purposes | ad 1.0 | l |

Stability of the formulations was determined after storage at temperatures of −20° C., +5° C. and +25° C. for 6 months. For this purpose, in each case 1 ml of said formulations was introduced into 2 ml clear glass vials and lyophilized therein. The vials were sealed with bromobutyl rubber stoppers and a flip-off flange cap.

Storage took place in a temperature- and humidity-monitored/controlled area with protection from light.

Table 2 depicts total contamination of the formulations in %.

TABLE 2

| | Time | | | |
|---|---|---|---|---|
| | T0 | T6 months | T6 months | T6 months |
| Temperature | | −20° C. | +5° C. | +25° C. |
| Example 1 | 1.704 | 1.7968 | 1.7563 | 1.8570 |
| Example 3 | 0.550 | 0.4808 | 0.4929 | 0.6714 |
| Example 5 | 0.563 | 0.4776 | 0.4932 | 0.5667 |
| Comparative Example 1 | 0.643 | 0.8872 | 1.9531 | 7.4321 |

The table indicates that the formulations of the invention are stable at −20° C., +5° C. and at +25° C. over a period of 6 months. At +5° C., total contaminations increase by no more than 0.0523% and at +25° C. by no more than 0.153%. In Comparative Example 1, total contaminations increase at +5° C. by 1.3101% and at +25° C. by 6.789%. The formulations of the invention are thus distinctly more stable than the formulation of the Comparative Example.

What is claimed is:

1. A formulation containing the peptide p277 or its variants having the structure Val-Leu-Gly-Gly-Gly-X₁-Ala-Leu-Leu-Arg-X₂-Ile-Pro-Ala-Leu-Asp-Ser-Leu-X₃-Pro-Ala-Asn-Glu-Asp (SEQ. I.D. No. 1), wherein X₁ and X₂ are each selected from Cys and Val and X₃ is selected from Thr and Lys, which formulation comprises a citrate buffer.

2. The formulation as claimed in claim 1, which comprises the p277 peptide (Val⁶-Val¹¹-Thr¹⁹) having the structure Val-Leu-Gly-Gly-Gly-Val-Ala-Leu-Leu-Arg-Val-Ile-Pro-Ala-Leu-Asp-Ser-Leu-Thr-Pro-Ala-Asn-Glu-Asp (SEQ. I.D. No. 2).

3. The formulation as claimed in claim 1, which further comprises an excipient selected from the group consisting of trehalose, maltose, lactose and mannitol.

4. The formulation as claimed in claim 3, which comprises mannitol.

5. The formulation as claimed in claim 4, wherein the mannitol content is from 5 mg/ml to 200 mg/ml.

6. The formulation as claimed in claim 4, wherein the mannitol content is from 25 mg/ml to 50 mg/ml.

7. The formulation as claimed in claim 4, wherein the mannitol content is about 40 mg/ml.

8. The formulation as claimed in claim 1, wherein the pH is from about pH 3 to about pH 6.

9. The formulation as claimed in claim 8, wherein the pH is from about pH 4.5 to about pH 6.

10. The formulation as claimed in claim 1, wherein the citrate buffer comprisess sodium citrate or potassium citrate.

11. The formulation as claimed in claim 1, wherein the citrate buffer comprisess hydrochloric acid.

12. The formulation as claimed in claim 1 which has been lyophilized.
